(19) United States
(12) Patent Application Publication
(10) Pub. No.: US 2024/0050203 A1
(43) Pub. Date: Feb. 15, 2024

(54) METHOD FOR MAKING A REMOTELY CUSTOM FIT DENTAL ORTHOTIC

(71) Applicant: Gregg Edward Ueckert, Austin, TX (US)

(72) Inventor: Gregg Edward Ueckert, Austin, TX (US)

(21) Appl. No.: 18/345,755

(22) Filed: Jun. 30, 2023

Related U.S. Application Data
(60) Provisional application No. 63/358,022, filed on Jul. 1, 2022.

(51) Int. Cl. A61C 13/00; A61C 9/00; A61C 13/34; B33Y 80/00

(52) U.S. Cl. CPC A61C 13/0004; A61C 9/0006; A61C 13/34; B33Y 80/00

(57) ABSTRACT

Methods for manufacturing remotely custom fit dental orthotics, where a wearer makes impressions of their upper and lower teeth and also makes a bite impression that determines the relationship of the upper teeth to the lower teeth, preferably in an orthopedically optimized position with respect to each other. The impressions and bite registration are sent by the wearer to a manufacturing facility in a different location. At the manufacturing facility, the impressions are used to make models of the wearer's upper and lower teeth. The models are mounted on an articulator, and the bite registration is used to position the mounted models in the orthopedically optimized position which is locked in to the articulator. The bite registration is removed and a dental orthotic customized to the wearer and the orthopedically optimized position is formed between the models. The custom orthotic is then sent to the wearer.

### CROSS-REFERENCE TO RELATED APPLICATIONS

This application claims a benefit of priority under 35 U.S.C. § 119(e) from U.S. Provisional Application No. 63/358,022, filed Jul. 1, 2022, entitled “METHOD FOR MAKING A REMOITELY CUSTOM FIT DENTAL ORTHOTIC,” which is fully incorporated by reference herein for all purposes.

### BACKGROUND

### Field of the Invention

The invention relates generally to manufacturing dental orthotics and more particularly to methods for manufacturing dental orthotics in which a user makes their own dental impressions and bite registrations and provides them to a manufacturing facility that then pours models using the provided impressions, mounts the working models on an articulator, sets the articulator in an orthopedically correct alignment and forms a custom-fit dental orthotic using the aligned articulator and working models.

### Related Art

Protective mouthpieces have long been used by athletes for the purpose of avoiding damage to their teeth. Conventionally, these mouthpieces have provided cushioning between a wearer's upper and lower teeth to reduce the effects of concussive impacts to the head and the jaw. The mouthpieces may also affect an athlete's performance and well-being by maintaining a desired position of the lower jaw with respect to the upper jaw.

The positioning of a person's jaw with respect to the skull can affect the person's head position and therefore the alignment of their vertebral postural chain in either a positive or negative fashion. Poor head position induces torque in the upper cervical spine, creating shear on the central nervous system (CNS) and limiting range of motion in the major joints of the body. This prevents optimal physiologic output and presents an increasing risk of injury through accommodation to the restricted movements and less than optimal physiologic alignment.

There are techniques that can be practiced on a person by trained medical professionals to determine an optimized, orthopedically aligned position of the person's jaw. Using these techniques, a custom-fit orthotic device can be formed and used to maintain the optimized, orthopedically aligned position of the jaw. These techniques, however, have a number of drawbacks. For instance, the availability of the trained medical professionals is limited, a great deal of time is commonly required to schedule and conduct a fitting, and the labor costs associated with the medical professionals are typically high. As a result, it is difficult to produce large numbers of dental orthotics in this manner, and widespread distribution of the orthotics is cost-prohibitive.

Consequently, the performance benefits of custom-fit dental orthotics are available to only a very limited number of people. It would therefore be desirable to provide methods for making custom-fit dental orthotics that are faster, less labor intensive, less costly, and more widely available than current methods.

### SUMMARY OF THE INVENTION

This disclosure is directed to systems and methods relating to the improvement of a person's orthopedic alignment, descending down the postural chain from the head to the feet by means of a custom-fit dental orthotic. (The user may alternatively be referred to herein as a wearer.) This device can improve physiologic performance and reduce injuries that are directly related to poor head position which causes poor alignment of the postural chain. The improvement of physiologic performance related to head position is discussed in, for example, U.S. Pat. Nos. 10,238,477, 10,675,127 and 11,045,344, which are incorporated herein by reference.

The improvement is achieved through the use of a fitting and manufacturing process that involves both the manufacturer of the dental orthotic and the user/wearer of the dental orthotic, who is in a location which is remote from the manufacturer (a manufacturing facility). “Remote” is used here to refer to locations that are not part of the manufacturing facility. For instance, the manufacturing facility may be in a first city, while the user/wearer may be in a different city, a different state, or even a different country. Wherever the user/wearer may be, it is not necessary for the manufacturer to provide personnel to perform the fitting process (i.e., making impressions of intended wearer's upper and lower teeth and taking an optimized bite registration).

In the embodiments disclosed herein, a wearer makes impressions of their upper and lower teeth and also makes a bite impression that identifies the relationship of the upper teeth to the lower teeth, preferably when the upper and lower teeth are in an orthopedically optimized position with respect to each other. These impressions and the bite registration are sent by the wearer to a manufacturing facility which is in a different location. At the manufacturing facility, the impressions are used to make models of the wearer's upper and lower teeth. The models are mounted on an articulator, and the bite registration is used to position the mounted models in the orthopedically optimized position. This position is locked in to the articulator so that the models can be returned to this position when the bite registration has been removed. With the bite registration removed and the models in the orthopedically optimized position, a dental orthotic is formed between the models. The orthotic is customized to the wearer and the bite position recorded by the bite registration. One or more additional steps (e.g., trimming the orthotic, labeling it, etc.) may be performed to finish the custom orthotic, and then the orthotic is sent to the wearer.

Because the dental impressions are taken by the user/wearer, it is not necessary to involve a medical professional in the fitting process, which reduces the time, expense and inconvenience of a professional fitting. This also allows the manufacturer to process larger numbers of orthotics, since it is not necessary for the manufacturer to provide personnel to perform the fitting. The orthotic nevertheless provides the benefits of the custom fit and the alignment of the user/wearer's jaw, namely improvement of the Cranio-Cervical/Cranio-Mandibular (CC/CM) relationship as compared to situations in which conventional mouthpieces are used, and the resulting improvements in the user/wearer's performance.

### BRIEF DESCRIPTION OF THE DRAWINGS

Other objects and advantages of the invention may become apparent upon reading the following detailed description and upon reference to the accompanying drawings.

FIG. 1 is a flow diagram illustrating an exemplary method for making a remotely custom-fit dental orthotic in accordance with some embodiments.

FIG. 2 is a flow diagram illustrating an exemplary method for a wearer to make dental impressions and a bite registration for a custom-fit dental orthotic in accordance with some embodiments.

FIG. 3 is a diagram, illustrating impressions of a wearer's upper and lower teeth and a bite registration in accordance with some embodiments.

FIG. 4 is a flow diagram illustrating an exemplary method for forming a bite registration with a formable mouthpiece in accordance with some embodiments.

FIG. 5 is a flow diagram illustrating an alternative method for forming a bite registration in accordance with some embodiments.

FIG. 6 is a diagram illustrating a bite fork and a bite registration made using a bite fork in accordance with some embodiments.

FIG. 7 is a flow diagram illustrating an exemplary method for manufacturing a custom dental orthotic at a manufacturing facility using remotely created dental impressions and a remotely created bite registration in accordance with some embodiments.

FIG. 8 is a diagram illustrating models of a wearer's upper and lower teeth in accordance with some embodiments.

FIG. 9 is a diagram illustrating an articulator with a mouthpiece-type bite registration positioned between upper and lower models of a wearer's teeth in accordance with some embodiments.

FIG. 10 is a diagram illustrating an articulator with a bite-fork-type bite registration positioned between upper and lower models of a wearer's teeth in accordance with some embodiments.

FIG. 11 is a diagram illustrating an articulator in orthopedically optimized position with the bite registration removed in accordance with some embodiments.

FIG. 12 is a diagram illustrating an articulator with an orthotic material molded around the teeth of the upper model in accordance with some embodiments.

FIG. 13 is a diagram illustrating an example of a custom dental orthotic molded to cover primarily the lower teeth in accordance with some embodiments.

While the invention is subject to various modifications and alternative forms, specific embodiments thereof are shown by way of example in the drawings and the accompanying detailed description. It should be understood, however, that the drawings and detailed description are not intended to limit the invention to the particular embodiment which is described. This disclosure is instead intended to cover all modifications, equivalents and alternatives falling within the scope of the present invention as defined by the appended claims. Further, the drawings may not be to scale, and may exaggerate one or more components in order to facilitate an understanding of the various features described herein.

### DETAILED DESCRIPTION OF EXEMPLARY EMBODIMENTS

One or more embodiments of the invention are described below. It should be noted that these and any other embodiments described below are exemplary and are intended to be illustrative of the invention rather than limiting.

As described herein, various embodiments of the invention comprise systems and methods for making custom-fit dental orthotics that maintain improved CC alignment, where the dental orthotics are made at a lab or other manufacturing facility, but the manufacturing process uses dental impressions and bite registrations that are made by and received from someone who is at a different location than the manufacturing facility, such as the user/wearer themselves.

In some embodiments, the user/wearer takes separate impressions of their upper and lower teeth. Additionally, the user/wearer takes an optimized bite registration of the upper and lower teeth when the lower jaw and Temporomandibular Joint (TMJ) are in an optimized position with respect to the upper jaw. Various techniques may be used to achieve the optimized position, such as gently tapping the teeth together, or using bite registration devices that are designed to gently move the lower jaw into an optimized position. The bite registration has impressions of the upper teeth on the top of the bite registration and impressions of the lower teeth on the underside of the bite registration. After the user/wearer has made the impressions and bite registration, they send them to the manufacturer.

The manufacturer receives the impressions and the bite registration and fabricates working models of the user/wearer's teeth. The impressions are effectively negatives of the teeth, and the models are positive replicas of the teeth. Separate models are made for the upper teeth and the lower teeth. Both working models are then mounted on a fully adjustable articulator. The bite registration is then placed between the upper and lower models and the positions of the models and the articulator are adjusted so that the teeth of the models are seated in the corresponding impressions in the bite registration. When the models are seated in the bite registration, they are in the same optimized position as the teeth when the bite registration was taken. This position is then locked into the articulator.

After the optimized position is locked into the articulator, the articulator is adjusted so that it can be moved and the bite registration is removed from between the mounted models of the wearer's teeth. A moldable material is then placed between the mounted models and the articulator is returned to the optimized position and locked in this position so that the opposing sides of the moldable material are molded to the shapes of the wearer's upper and lower teeth (the upper and lower models). When the moldable material hardens, it forms a dental orthotic which will not only protect the wearer's teeth, but will also hold the wearer's lower jaw in an orthopedically optimized position that will improve the wearer's performance.

In the example above, the orthotic is formed by placing a moldable material between the models mounted on the articulator and returning the articulator to the optimized position. In an alternative embodiment, the manufacturer may use a heat and pressure moldable material (e.g., Ethyl Vinyl Acetate, or EVA) to form an unfinished dental orthotic on the user/wearer's working model. The unfinished orthotic may be an upper or lower orthotic, as selected by the user/wearer. (The orthotic could also be both upper and lower, although these are typically disfavored, since they can obstruct the wearer's airflow.) An unfinished upper orthotic can be formed on the upper model, and an unfinished lower orthotic can be formed on the lower model. The orthotic could alternatively be 3-D printed from a digital scan of the impressions or working models.

After the optimized position has been locked into the articulator, the biting surface of the unfinished orthotic is softened (e.g., by heating the surface) and placed on the corresponding model (i.e., the model on which it was formed) on the articulator, and then the articulator is returned to the optimized position. This causes the second model (i.e., the model other than the one on which the unfinished orthotic was formed) to make corresponding impressions on the second side of the unfinished orthotic. These impressions are positioned so that when the wearer's teeth engage the impressions in the orthotic, the wearer's lower jaw is placed in the orthopedically optimized position. After the impressions are made in the second side of the unfinished orthotic, the orthotic is removed from the articulator, finished, and sent to the wearer.

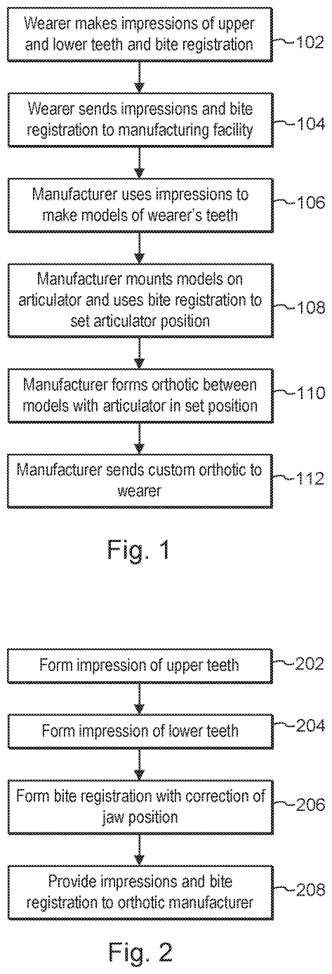

Referring to FIG. 1, a flow diagram illustrating an exemplary method for making a remotely custom-fit dental orthotic in accordance with some embodiments is shown. At step 102, a wearer makes impressions of their upper teeth and lower teeth, and also makes a bite registration which records the relative position of the lower teeth with respect to the upper teeth. The wearer then sends the impressions and bite registration that they have made to the manufacturing facility (step 104).

When the manufacturing facility has received the impressions and bite registration, it proceeds to make models of the wearer's upper and lower teeth using the impressions (step 106). The models of the wearer's upper and lower teeth are then mounted on a fully adjustable articulator that allows the models to articulate in the same way the wearer's jaw articulates the teeth (step 108). The bite registration is used to adjust the position of the articulator so that the upper and lower models mimic the positions of the wearer's teeth and jaw (id.) Preferably, the bite registration records an optimized position of the lower jaw with respect to the upper jaw/skull. The optimized position is locked in to the articulator.

The bite registration is removed from the articulator and a custom orthotic is formed between the upper and lower models with the articulator in the optimized position (step 110). The custom orthotic is therefore custom-fit to the wearer's teeth without the manufacturer or any trained technicians having to perform a fitting, or even be in the same location as the wearer. The custom orthotic formed on the articulator is removed from the articulator and is sent to the wearer (step 112).

Referring to FIG. 2, a flow diagram illustrating an exemplary method for a wearer to make dental impressions and a bite registration for a custom-fit dental orthotic in accordance with some embodiments is shown. In steps 202 and 204, a user forms impressions of their upper and lower teeth. Each impression can be made, for example, by preparing a volume of dental putty, placing the putty in a tray, placing the tray with the putty in the user's mouth, and pressing the putty-filled tray upward or downward against the user's teeth. The dental putty can for example, be a two-part addition-reaction silicone (hydrophilic vinylpolysiloxane) material. The two parts of the material are mixed together to form the dental putty and placed on a dental tray. The tray is then placed in the wearer's mouth and pressed toward the upper or lower teeth so that the material conforms to the teeth and gums. The putty is then allowed to harden, and the tray and putty are pulled off of the user's teeth, leaving an impression of the user's teeth in the putty. Ideally, the impression covers all of the exposed surfaces of the teeth.

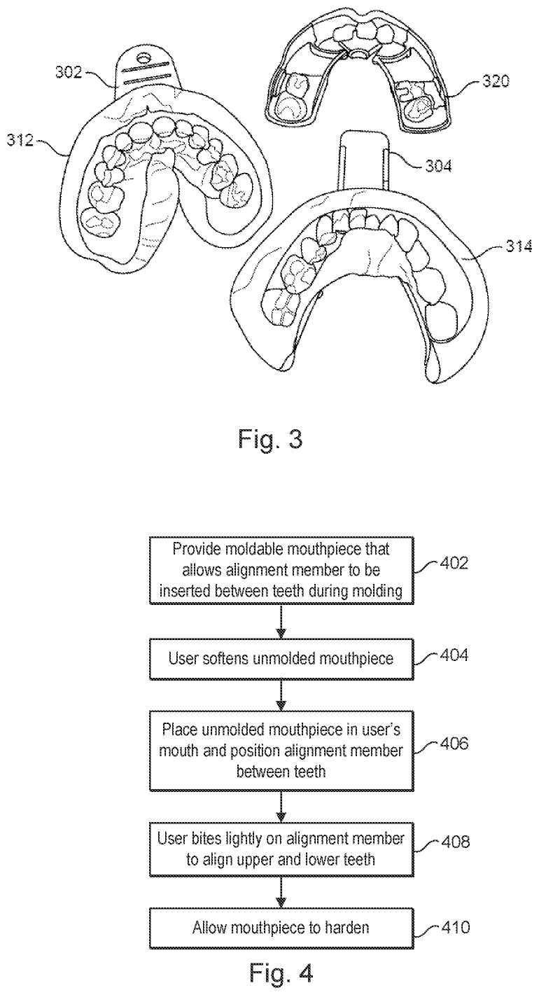

Referring to FIG. 3, impressions of the user's upper and lower teeth are shown. In this figure, the dental putty in tray 302 has been pressed onto the user's upper teeth to form impression 312. Similarly, dental putty in tray 304 has been pressed onto the user's lower teeth to form impression 314.

Referring again to FIG. 2, in addition to forming impressions of the upper and lower teeth, the user forms a bite registration (206). The bite registration is similar to the upper and lower dental impressions, in that it has impressions of the biting surfaces of the teeth. Rather than having indentations of only the upper teeth, or only the lower teeth, the bite registration has indentations of both upper and lower teeth while the user's jaw is in an optimized position with respect to the skull (i.e., with the lower teeth in an optimized position with respect to the upper teeth). The bite registration does not need to cover all of the exposed surfaces of the teeth—it simply needs to have sufficient impressions to define the optimized position of the jaw with respect to the skull (i.e., the position of the lower teeth with respect to the upper teeth). When the impressions and bite registration have been made by the user, they can be sent by the user to a lab or other manufacturing facility (208) for use in making a custom-fit dental orthotic.

Referring again to FIG. 3, an example of a bite registration 320 is shown. In this case, the bite registration comprises a formable mouthpiece, which is similar to conventional protective mouth guards, except that bite registration 320 has a front portion which is molded to the front teeth (upper and lower incisors) and rear portions which are molded to the rear teeth (upper and lower molars), with gaps between the front and rear portions. The gaps allow an alignment member to be positioned between the upper and lower teeth (at the canines), where the alignment member contacts the upper and lower teeth and urges them into an optimized relationship. This is described in detail in U.S. Pat. No. 11,045,344, which is included herein by reference. The portion of the bite registration which is positioned between the upper and lower molars may be thick enough, or may have structural properties that keep the upper and lower molars at least a minimum distance from each other. This minimal spacing may be provided to ensure that there will be sufficient cushioning material between the molars in the finished orthotic, and/or to ensure that the molars feel sufficient initial pressure through the orthotic to be sure there is proper proprioception.

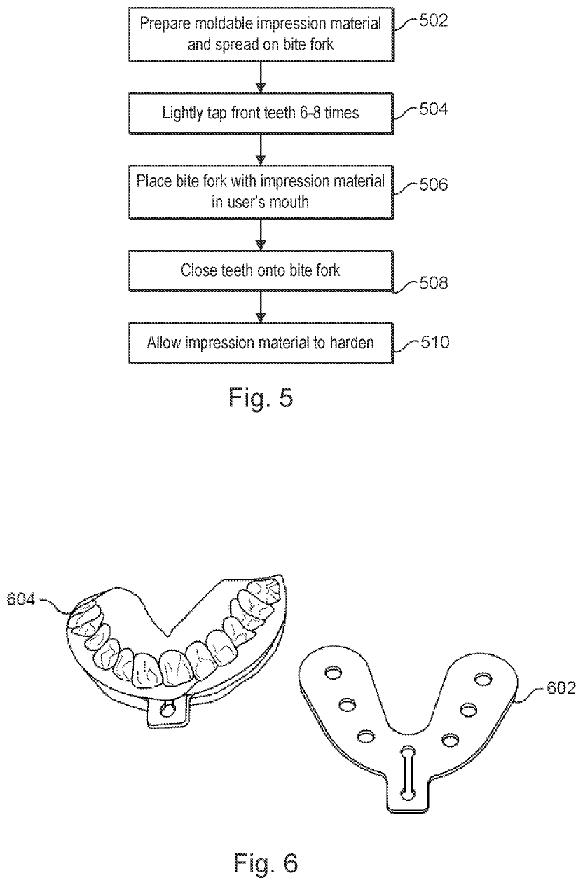

Forming the bite registration with the user's jaw in in an optimized position (see step 106 of FIG. 1) can be accomplished in a number of different ways. Two exemplary methods for forming the bite registration are illustrated in FIGS. 4 and 5.

Referring to FIG. 4, an exemplary method for forming a bite registration with a formable mouthpiece is shown. In this example, a moldable mouthpiece that can be formed while maintaining an optimized jaw/skull relationship is provided (402). The mouthpiece is made using a thermoplastic material that can be softened by placing the mouthpiece in hot water for a short period of time. When the mouthpiece is removed from the hot water, it gradually hardens. After the user softens the unmolded mouthpiece (404), the mouthpiece in the user's mouth and an alignment member is positioned between the user's teeth (406). The user lightly bites down on the mouthpiece so that the user's upper and lower teeth make indentations in the moldable material of the mouthpiece (408). As the user bites down on the mouthpiece, the user's teeth come into contact with the alignment member, urging the users jaw into an optimized position with respect to the skull. The mouthpiece may also include a substrate or other structure which maintains a minimum spacing between the teeth. The spacing may range from about 2 mm to about 5 mm, but in a preferred embodiment, the minimum spacing is 3 mm. This relationship is recorded by the indentations of the teeth in the mouthpiece. With the jaw in this optimized position, the mouthpiece is allowed to harden (410).

Referring to FIG. 5, an alternative method for forming a bite registration is shown. In the example of FIG. 5, the bite registration is formed using a “bite fork”. the bite fork is, in some embodiments, simply a flat plate that is generally horseshoe-shaped, so that when it is positioned in a user's mouth, the plate forms a barrier between the user's upper and lower teeth. In the method of FIG. 5, moldable impression material such as dental putty is placed on both the upper and lower sides of the bite fork (502). The bite fork has holes in it so that the dental putty flows through the holes, connecting the putty on the upper side of the bite fork to the putty on the lower side of the bite fork. In this method, no alignment number is used to align the user's jaw to their skull. Instead, the user lightly taps their front teeth together (e.g., 6 to 8 times) (504). This is done to relax the jaw, allowing it to naturally move into an optimized position with respect to the skull. The bite fork with the dental putty is then placed in the user's mouth (506) and the user lightly bites down on the bite fork (508). In some embodiments, the bite fork has a thickness that ensures a minimum spacing (e.g., 3 mm) between the teeth as the user bites down on the bite fork. The moldable material is then allowed to harden with the jaw in this optimized position (510). Referring to FIG. 6, examples of a bite fork (602) and a bite registration (604) which is made using a bite fork are shown.

The dental impressions and bite registration can be done without difficulty, and these steps are performed by a user in their own home or other location at their convenience. In some embodiments, a kit with all the necessary materials (e.g., impression trays, dental putty and mouthpiece or bite fork) can be provided to a user with instructions for forming the impressions and bite registration, and these can be completed without the help of a trained technician or medical professional. The kit provided to the user may even include materials for packaging and shipping the impressions and bite registration to the manufacturing facility.

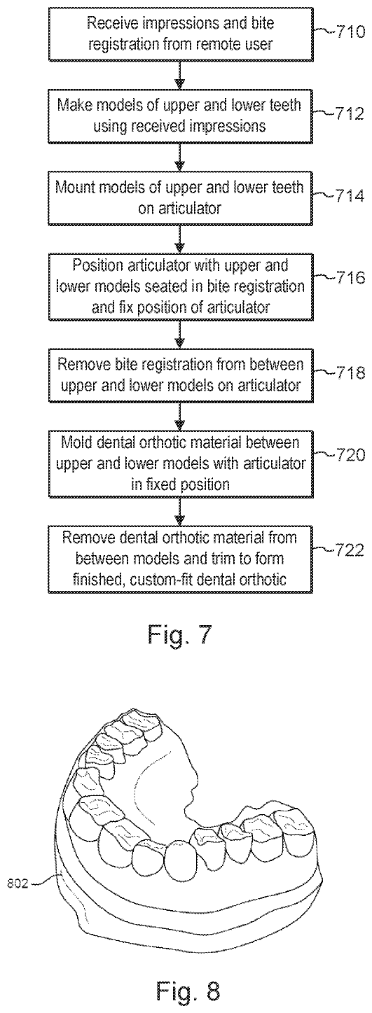

When the impressions and bite registration have been made by the user and sent to a lab or other manufacturing facility, they can be used by the manufacturing facility to manufacture a custom-fit dental orthotic. Referring to FIG. 7, a flow diagram is shown to illustrate an exemplary method for manufacturing a custom dental orthotic at a manufacturing facility using remotely created dental impressions and a remotely created bite registration in accordance with some embodiments. The dental impressions and bite registration are received by the manufacturing facility (710) and the facility makes models of the user's upper and lower teeth (712). An example of the models is shown in FIG. 8. In this figure, the illustrated model 802 is a model of the wearer's upper teeth.

The models of the user's upper and lower teeth are then mounted on an articulator (714). The articulator allows the models to be moved in a manner similar to the motion of a user's jaw and can be adjusted to match the specific bite of a user. After the models are mounted on the articulator, the articulator is loosened so that the models (which are still secured to the articulator) can be moved with multiple degrees of freedom (roll, pitch and yaw, in addition to translation toward each other). The bite registration is then positioned between the upper model and the lower model, and each model is seated in the corresponding indentations of the bite registration (716). In other words, the teeth of the upper model are seated in the indentations made by the user's upper teeth and the teeth of the lower model are seated in the indentations made by the user's lower teeth.

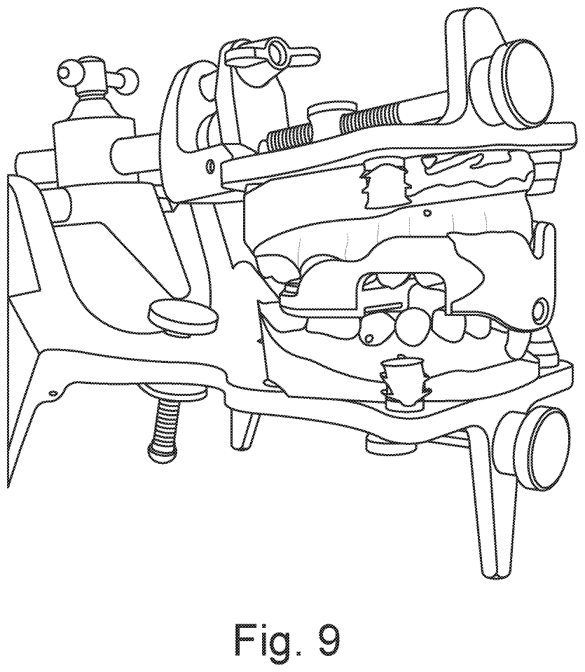

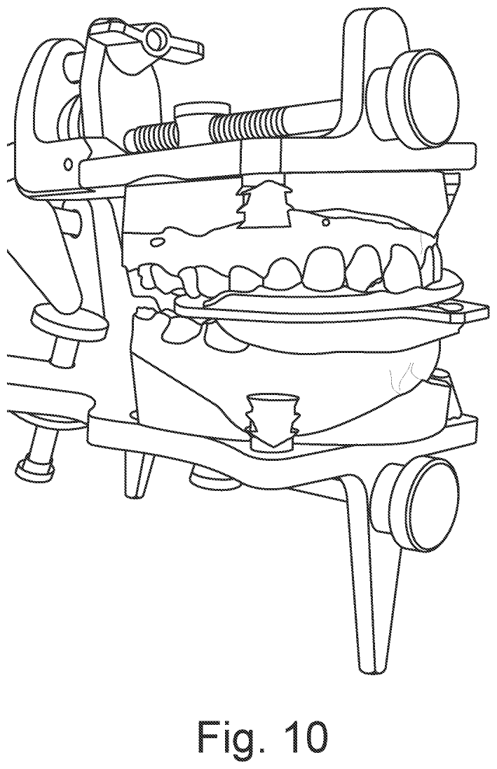

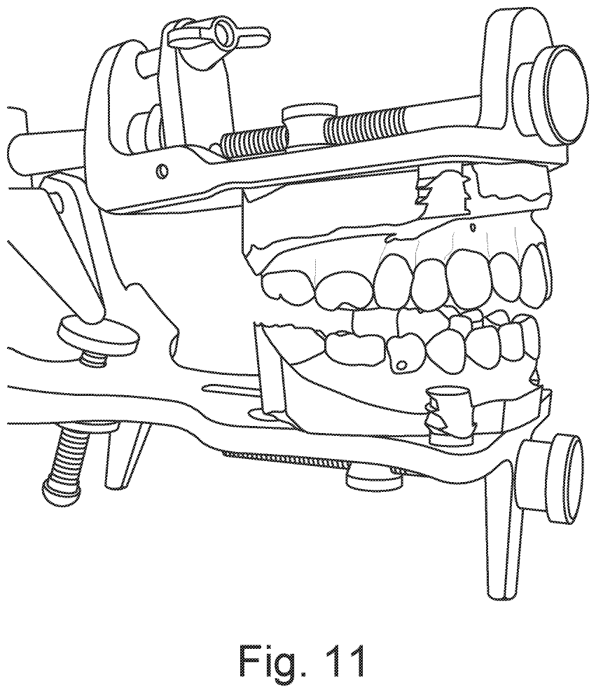

With the teeth of the upper and lower models seated in the indentations of the bite registration, the articulator is tightened (716) to “fix” this position so that the articulator can be returned to exactly this position when the bite registration is removed. The bite registration is then removed from between the models (718) and the articulator is returned to the fixed position. FIG. 9 shows the articulator in the fixed position with the mouthpiece-type bite registration still positioned between the upper and lower models. FIG. 10 shows the articulator in the fixed position with a bite-fork-type bite registration positioned between the upper and lower models. FIG. 11 shows the articulator in the fixed position with the bite registration removed.

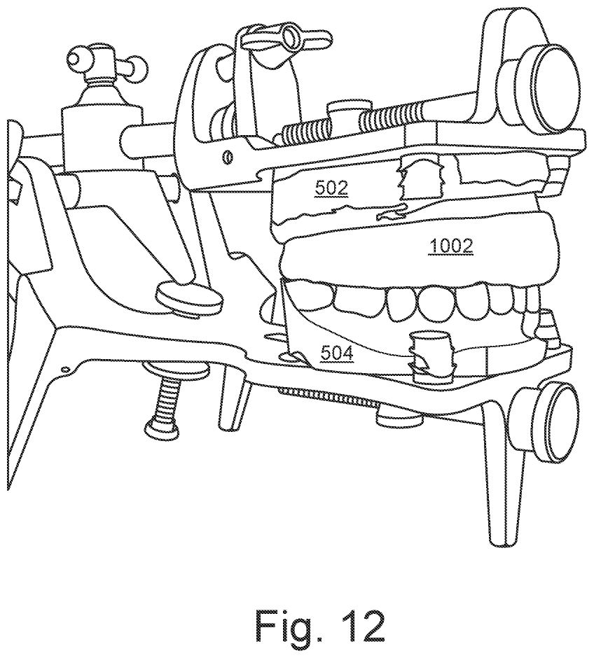

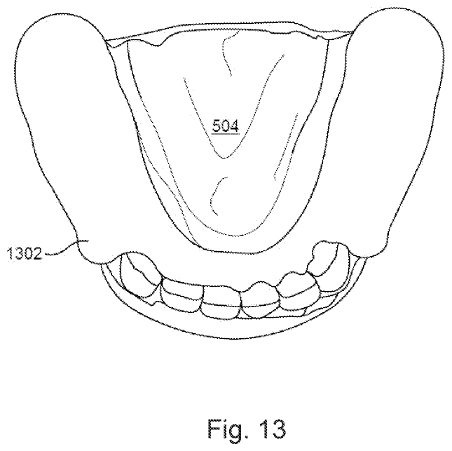

It can be seen in FIG. 11 that there is a gap between the upper model and the lower model which was previously occupied by the bite registration. As noted above, the gap will preferably provide a minimum spacing of at least 3 mm between the models. with the articulator in this fixed position, a semi-elastic material is injected between the upper and lower models (720) to form the rough shape of the dental orthotic. FIG. 12 shows the articulator with the orthotic material molded around the teeth of the upper model. It should be noted that while the orthotic in this example is formed primarily around the upper model, the orthotic material covers at least apportionment of the lower model including the upper, biting surfaces of the teeth so that the positioning of the lower teeth (and jaw) will be maintained with respect to the upper teeth (and skull). In alternative embodiments, the orthotic may be molded to cover most of both the upper and lower teeth, or to cover primarily the lower teeth (with indentations corresponding to the upper teeth). An example of an orthotic 1302 molded to cover primarily the lower teeth is shown in FIG. 13.

Referring again to FIG. 7, when the material hardens, it is removed from the models and is trimmed and finished to make the final, custom-fit dental orthotic. This final dental orthotic is then shipped back to the user, who performed the initial fitting to produce the dental impressions and bite registration (722).

As noted above, by providing fitting materials to the end user/wearer of the orthotic to have them make their own dental impressions and bite registration which are then sent back to the manufacturing facility, the present methods reduce the time, expense and inconvenience of having to provide a medical professional or technician conduct the fitting. This also allows the manufacturer to process larger numbers of orthotics to provide to larger numbers of people the benefits of the custom fit and the alignment of the users/wearers' jaws, namely concussion protection and performance improvement.

It should be noted that there may be many variations of the foregoing embodiments that are within the scope of the claims below. For example, one embodiment comprises a method for producing a remotely custom-fit dental orthotic. This method includes receiving, at a manufacturing location, upper and lower impressions of a wearer's teeth and a bite registration of the wearer's teeth, where the upper and lower impressions and the bite registration were created at a second location which is remote from the manufacturing location. Models of the wearer's upper and lower teeth are then created using the upper and lower impressions. The models are mounted on an articulator and a position of the articulator is set using the mounted models and the bite registration, where each of the mounted models is mated with the corresponding impression of the bite surfaces of the teeth in the bite registration. The bite registration is then removed from between the mounted models and a custom dental orthotic is formed between the mounted models with the articulator in the set position.

In some embodiments, forming the custom dental orthotic comprises molding a moldable material between the mounted models, allowing the moldable material to harden, and trimming the hardened moldable material.

In some embodiments, creating the models comprises pouring fluid molding material into the upper and lower impressions and allowing the fluid molding material to harden into the models. In other embodiments, creating the models may comprise 3-D scanning the upper and lower impressions, and generating 3-D prints complementary to the upper and lower impressions, where the models comprise the 3-D prints.

In some embodiments, the position in which the articulator is set comprises an orthopedically optimized position for the wearer. The method may include creating the bite registration by the wearer when the wearer's teeth and jaw are in the orthopedically optimized position. In some embodiments, this is accomplished by softening bite registration material (e.g., a formable mouthpiece), gently tapping the wearer's teeth together multiple (e.g., 6-8) times, placing the bite registration material between the wearer's teeth, biting down on the bite registration material to form impressions of biting surfaces of the upper and lower teeth in the bite registration material, and allowing the bite registration material to harden. In other embodiments, rather than tapping the wearer's teeth together, a positioning aid is applied to the wearer's teeth to urge the wearer's jaw toward the orthopedically optimized position. For instance, a dowel-like alignment member may be placed between the upper and lower canines or bicuspids to urge the lower jaw into a preferred alignment with the upper jaw.

In some embodiments, the upper and lower impressions and the bite registration are created at a second location (e.g., the location of the wearer) which is different than the location of the manufacturing facility. A custom fitting kit may be sent from the manufacturing location to the second location, where the custom fitting kit includes materials for making the upper and lower impressions and the bite registration. The kit may include, for example, dental trays and a moldable dental putty for making the upper and lower impressions. The kit may also include materials for making the bite registration, such as a bite fork and a moldable dental putty, or a formable mouthpiece which is adapted to be softened by heating the mouthpiece.

An alternative embodiment comprises a method for producing multiple remotely custom-fit dental orthotics (e.g., for different customers). In this method, multiple sets of custom fitting elements are received at a manufacturing location, each set corresponding to a different wearer at a different wearer location. Each set of custom fitting elements includes upper and lower impressions of the corresponding wearer's teeth and a bite registration of the corresponding wearer's teeth. For each received set of custom fitting elements, the manufacturing facility creates models of the corresponding wearer's upper and lower teeth using the upper and lower impressions and mounts the models on an articulator. The manufacturing facility sets a first position of the articulator using the mounted models and the bite registration, where each of the mounted models is mated with a corresponding impression in the bite registration. The bite registration is then removed from between the mounted models and a custom dental orthotic is formed between the mounted models with the articulator in the first position. The custom dental orthotic is then returned to the corresponding wearer.

Another alternative embodiment comprises a method for performing a custom fitting for a custom dental orthotic that will be manufactured remotely from the fitting location. In this method, a kit for custom fitting a dental orthotic is obtained at a wearer's location. The kit includes materials for making an upper impression of the wearer's upper teeth, a lower impression of the wearer's lower teeth, and a bite registration that has impressions of bite surfaces of both the wearer's upper teeth and lower teeth. The upper impression, the lower impression and the bite registration are then made at the wearer's location, and the upper impression, the lower impression and the bite registration are sent from the wearer's location to a manufacturing facility.

In some embodiments, the materials for making the upper impression and lower impression each comprises comprising a dental tray and a moldable dental putty, and the materials for making the bite registration comprise either a bite fork and a moldable dental putty, or a formable mouthpiece which is adapted to be softened by heating the mouthpiece. In some embodiments, the bite registration is made while the wearer's teeth (and jaw) are in an orthopedically optimized position. In some cases, the wearer's teeth may be placed in the orthopedically optimized position by gently tapping the wearer's teeth together a number of times (e.g., 6-8 times) to relax the jaw so that it moves to the orthopedically optimized position. In other cases, the wearer's teeth may be placed in the orthopedically optimized position by applying a positioning aid to the wearer's teeth to urge the wearer's jaw toward the orthopedically optimized position.

Many other variations are also possible within the scope of the invention.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the claims. As used herein, the terms “comprises,” “comprising,” or any other variations thereof, are intended to be interpreted as non-exclusively including the elements or limitations which follow those terms. Accordingly, a system, method, or other embodiment that comprises a set of elements is not limited to only those elements, and may include other elements not expressly listed or inherent to the claimed embodiment.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention as detailed within the following claims.

What is claimed is:

1. A method for producing a remotely custom-fit dental orthotic:
receiving, at a manufacturing location, upper and lower impressions of a wearer's teeth and a bite registration of the wearer's teeth, the upper and lower impressions and the bite registration created at a second location which is remote from the manufacturing location;
creating models of the wearer's upper and lower teeth using the upper and lower impressions;
mounting the models on an articulator;
setting a first position of the articulator using the mounted models and the bite registration, wherein each of the mounted models is mated with a corresponding impression in the bite registration;
removing the bite registration from between the mounted models; and
forming a custom dental orthotic between the mounted models with the articulator in the first position.

2. The method of claim 1, wherein forming the custom dental orthotic comprises molding a moldable material between the mounted models, allowing the moldable material to harden, and trimming the hardened moldable material.

3. The method of claim 1, wherein creating the models comprises pouring fluid molding material into the upper and lower impressions and allowing the fluid molding material to harden into the models.

4. The method of claim 1, wherein creating the models comprises 3-D scanning the upper and lower impressions, and generating 3-D prints complementary to the upper and lower impressions, the models comprising the 3-D prints.

5. The method of claim 1, wherein the first position comprises an orthopedically optimized position for the wearer.

6. The method of claim 5, further comprising creating the bite registration with the wearer's teeth in the orthopedically optimized position.

7. The method of claim 6, further comprising placing the wearer's teeth in the orthopedically optimized position by softening a bite registration material, tapping the wearer's teeth together a plurality of times, placing the bite registration material between the wearer's teeth, biting the bite registration material to form impressions of biting surfaces of the upper and lower teeth in the bite registration material, and allowing the bite registration material to harden.

8. The method of claim 6, further comprising placing the wearer's teeth in the orthopedically optimized position by softening a bite registration material, placing the bite registration material between the wearer's teeth, applying a positioning aid to the wearer's teeth to urge the wearer's jaw toward the orthopedically optimized position, biting the bite registration material to form impressions of biting surfaces of the upper and lower teeth in the bite registration material, and allowing the bite registration material to harden.

9. The method of claim 1, wherein each impression comprises a tray holding moldable material, the moldable material molded around the wearer's teeth and hardened to form a negative of the wearer's teeth.

10. The method of claim 1, wherein removing the bite registration from between the mounted models comprises adjusting one or more controls of the articulator to allow the models to be moved apart, removing the bite registration from between the mounted models, returning the articulator to the first position, and adjusting the one or more controls of the articulator to fix the articulator in the first position.

11. The method of claim 1, further comprising, prior to receiving the upper and lower impressions and the bite registration, sending from the manufacturing location to the second location a custom fitting kit including materials for making the upper and lower impressions and the bite registration.

12. The method of claim 11, the materials for making the upper and lower impressions comprising dental trays and a moldable dental putty.

13. The method of claim 11, the materials for making the bite registration comprising a bite fork and a moldable dental putty.

14. The method of claim 11, the materials for making the bite registration comprising a formable mouthpiece which is adapted to be softened by heating the mouthpiece.

15. The method of claim 1, further comprising, at the second location, creating the upper and lower impressions and the bite registration.

16. A method for producing remotely custom-fit dental orthotics:
receiving, at a manufacturing location, a plurality of sets of custom fitting elements,
each set corresponding to a different wearer at a different wearer location
each set comprising upper and lower impressions of the corresponding wearer's teeth and a bite registration of the corresponding wearer's teeth; and
for each received set of custom fitting elements:
creating models of the corresponding wearer's upper and lower teeth using the upper and lower impressions,
mounting the models on an articulator,
setting a first position of the articulator using the mounted models and the bite registration, wherein each of the mounted models is mated with a corresponding impression in the bite registration,
removing the bite registration from between the mounted models,
forming a custom dental orthotic between the mounted models with the articulator in the first position, and
returning the custom dental orthotic to the corresponding wearer.

17. A method for custom-fitting for a remotely manufactured dental orthotic:
obtaining, at a wearer's location, a kit for custom-fitting a dental orthotic, the kit including materials for making an upper impression of the wearer's upper teeth, a lower impression of the wearer's lower teeth, and a bite registration including impressions of bite surfaces of the wearer's upper and lower teeth;
making the upper impression, the lower impression and the bite registration; and
sending the upper impression, the lower impression and the bite registration to a manufacturing facility.

18. The method of claim 17:
wherein the materials for making the upper impression and lower impression each comprises comprising a dental tray and a moldable dental putty; and
wherein the materials for making the bite registration comprise either: a bite fork and a moldable dental putty; or a formable mouthpiece which is adapted to be softened by heating the mouthpiece.

19. The method of claim 17, wherein making the bite registration comprises making the bite registration while the wearer's teeth are in an orthopedically optimized position.

20. The method of claim 19, further comprising placing the wearer's teeth in the orthopedically optimized position by:
softening a bite registration material;
either tapping the wearer's teeth together a plurality of times, or applying a positioning aid to the wearer's teeth to urge the wearer's jaw toward the orthopedically optimized position;
placing the bite registration material between the wearer's teeth;
biting the bite registration material to form impressions of biting surfaces of the upper and lower teeth in the bite registration material; and
allowing the bite registration material to harden.
